(19) United States
(12) Patent Application Publication
(10) Pub. No.: US 2015/0291779 A1
(43) Pub. Date: Oct. 15, 2015

(54) COTTON BUD

(71) Applicant: ROWEG HOLDING AG, Erlenbach ZH (CH)

(72) Inventors: Beat Häni, Zuzwil (CH); Christian Rytka, Wehr (DE); Guy Petignat, Erlenbach (CH)

(73) Assignee: Roweg Holding AG, Erlenbach ZH (CH)

(21) Appl. No.: 14/441,210

(22) PCT Filed: Nov. 11, 2013
(86) PCT No.: PCT/CH2013/000195

§ 371 Date: May 7, 2015

(30) Foreign Application Priority Data
Nov. 13, 2012 (CH) 02367/12

(51) Int. Cl. C08L 1/02; C08L 101/16; A23G 9/50; C06F 5/00; A61F 13/38; A23G 3/56

(52) U.S. Cl. CPC C08L 1/02; A61F 13/38; A23G 3/563; A23G 9/503; C06F 5/00; C08L 101/16; A61F 13/385

(57) ABSTRACT

A cotton bud with a rod-like grip body and with a bud body that is arranged at least at one free end of the grip body. The rod-like grip body is a biologically degradable extrusion body.

### BACKGROUND OF THE INVENTION

1. Field of the Invention

The invention lies in the field of hygiene products and care products and relates to a cotton bud (cotton swab), with a rod-like grip body and with a bud body arranged at least at one free end of the grip body.

2. Description of Related Art

Cotton buds of the initially mentioned type and with bud bodies fastened at both ends are used in a multitude of manners for daily beauty care, in particular for cleaning the ears, with eye cosmetics, manicure, lipstick correction, etc.

With regard to the cotton buds, it is the case of so-called disposable articles, which are led to waste treatment after having been used once. It is therefore desirable for the cotton bud, as a whole or at least in part, to be biologically degradable and moreover as a whole or at least in part, to also be manufactured of renewable raw materials. Whereas the bud body as a rule consists of natural fibers, such as cotton, the grip body is often manufactured of a plastic which is not biologically degradable, such as polypropylene.

A further demand on such a cotton bud is its inexpensive manufacture. Since with regard to the cotton bud, it is the case of a disposable article, this should be as inexpensive as possible. The price of the cotton bud is composed of the raw material costs as well as of the manufacturing expense. It is in particular the manufacture of the grip body which is at the forefront with regard to the manufacturing costs.

It is therefore not too surprising that grips bodies of plastic which is biologically non-degradable and thus less environmentally friendly, but inexpensive instead, are widespread as was hitherto the case. Even the manufacture of the grip bodies from plastic requires comparatively little effort and is therefore inexpensive. Thus, for example, DE-A-2 013 886 describes a cotton bud with a grip body of a plastic tube piece which is manufactured with an extrusion method.

Earlier variants of cotton buds of wood are no longer obtainable today. This is due to the fact that the grip body, on account of the application of such care products in eye cosmetics, should not be too rigid or stiff. The grip bodies used nowadays, although still being comparatively stiff, however have a certain pliability or flexibility, so they buckle when a certain bending force is exceeded, but without splintering. The risk of injury with an undesired incorrect manipulation during the application of make-up or during the cleaning of the ears, for example, is reduced on account of this.

DE-U-297 16 819 describes a compostable cotton bud that is manufactured of cardboard or paper. The grip body of cardboard is manufactured by way of this being cut out or punched from a two-dimensional cardboard body, or is obtained by way of a cellulose pulp being filled into suitable casting moulds and being pressed. The grip body of paper is manufactured as a wound body, with which paper strips are wound into a rod-like body with suitable winding techniques or technology.

The grip bodies described above either have the disadvantage that they are not environmentally friendly or that their manufacture requires some effort and is accordingly expensive.

### SUMMARY OF THE INVENTION

It is therefore the object of the invention, to suggest a cotton bud of the previously mentioned type, which on the one hand is inexpensive to manufacture, but on the other hand comprises a biologically degradable (biodegradable) grip body.

The cotton bud preferably comprises a rod-like grip body that is designed as a biologically degradable extrusion body. The grip body preferably includes a material matrix as well as fibers, in particular natural fibers. The fibers preferably serve as reinforcement fibers. The grip body preferably comprises fibers that are partly or completely embedded into the material matrix.

The grip body can, for example, include cellulose fibers. Thereby, all or only a part of the fibers of the grip body can be cellulose fibers. The cellulose fibers can be obtained, for example, from waste paper, directly from wood and/or rice husks, straw and/or cereal husks.

According to a preferred further development of the invention, the grip body has a core, in particular a reinforcement core, which is enveloped by the material matrix. The material matrix according to this embodiment preferably includes fibers, such as natural fibers and particularly biodegradable fibers. The type and characteristics of the fibers of the material matrix can correspond to the fibers that are disclosed further below in the context of the material matrix, and its characteristics.

The reinforcement core according to a preferred embodiment is or includes a longitudinal (elongate) body, in particular a textile longitudinal body. The textile longitudinal body comprises or consists preferably of fibers, in particular natural fibers, which are moreover preferably biologically degradable.

The reinforcement core particularly preferably includes a linear, textile formation or a yarn, or consists of this. The linear textile formation can be a spun yarn. Twisted yarn, threads or cords as a textile formation are likewise not to be ruled out. The reinforcement core can moreover be a filament, for example, of a biologically degradable plastic.

The reinforcement core can also include a tube-like or flexible-tube-like, textile longitudinal body or consist thereof The tubular or hose-like textile longitudinal body can be designed as a braided fabric, woven fabric, knitted fabric, crocheted fabric, non-woven or felt.

The type and characteristics of the fibers of the textile reinforcement core can correspond to the fibers and their characteristics, which have already been disclosed or are disclosed hereinafter in the context of the material matrix.

The textile reinforcement core can be impregnated partly or completely with a material which, for example, after its curing or solidification creates an interconnection) between the fibers and intrinsically stiffens the textile reinforcement body. The impregnation of the reinforcement body can also be effected by way of the material matrix itself, which, for example, during the extrusion process penetrates into the textile reinforcement body and/or attaches itself to the surface of the reinforcement core.

The reinforcement core can, for example, have a diameter of 0.1 to 3 mm, preferably of 0.2 to 2 mm, and in particular of 0.5 to 1.5 mm

The reinforcement core has the function of a reinforcement. The term “reinforcement” in this context means that the reinforcement core absorbs or accommodates tensile forces, bending forces and/or compression forces acting upon the grip body. For example, it prevents the grip body from prematurely breaking with tensile or bending loading. The reinforcement core moreover also prevents parts of the grip body from falling apart on buckling or breakage. This means that a grip body with reinforcement core has a greater resistance to breakage and elongation at break than a grip body with a similar construction, but without a reinforcement core.

If the material matrix comprises fibers, then the material matrix in particular serves as a binding agent which holds together the fibers and forms a fibre-reinforced structure with these fibers.

The material matrix comprises or consists preferably of one of the subsequently specified, biologically degradable materials. Moreover, the material matrix can also consist of a mixture of these materials or comprise this mixture:

starch, starch derivatives, gluten, hemicellulose, sugar, lignin, sorbitol, chitin, gelatine, proteins, wax, glycerine, fructose, methylcellulose, modified methylcellulose, cellulose derivatives, agar-agar, pectin and/or biologically degradable polymers.

Biologically degradable polymers can be:

biologically degradable copolyesters, such as BTA, in particular blends with further biologically degradable polymers,

thermoplastic starch (TPS), starch blends,

polylactide or polylactic acid (PLA), and PLA blends,

cellulose products such as cellulose acetate (CA),

polyhydroxyalkanoates (PHA), polyhydroxybutyric acid (PHB).

biologically degradable polymers based on mineral oil such as polycaprolactone (PCL),

biologically degradable polymers which are derived chemically from lignin, chitin, casein, gelatine, proteins generally or vegetable oils,

biologically degradable polymers based on modified cellulose derivatives.

The material matrix can moreover be formed by way of dissolving the fibers, in particular the natural fibers, with the help of solvents, so that self-reinforcing cellulose composites arise.

The material matrix in particular serves for the positive-fit, chemical and/or physical cross-linking of the fibers, in particular of the natural fibers, or for the formation of hydrogen bridges or other bonds, which contribute to an improved coherence of the material.

The share of the material matrix with regard to the volume can be greater, equal or smaller that the share of fibers. If the share of fibers is significantly predominant, then the fibers can even form a fiber mass, whose individual fibers are bonded to one another via the material matrix acting as a binding agent. The material matrix as a result, together with the fibers forms a fibre-reinforced structure.

The material matrix can be added separately or be already contained in the starting material, so that its function as a binding agent merely has to be activated during the manufacturing process. This is particularly the case if the material matrix, for example, includes starch, sugar or lignin.

The grip body preferably comprises 10% by volume or more, in particular 30% by volume or more and advantageously 40% by volume or more, of fibers, in particular natural fibers. Moreover, the grip body preferably comprises 90% by volume or less, in particular 80% by volume or less, and advantageously 70% by volume or less, of fibers, in particular of natural fibers. The fibers are preferably secondary fibers of recycled, i.e. reused fiber material. The fibers can, for example, be cellulose fibers as has already been mentioned. The recycled starting material is preferably a biological fiber material which can be obtained very economically and which is obtained e.g. from waste paper or from waste from rice production.

Cellulose fibers can e.g. be obtained from ground or frayed waste-paper, i.e. waste-paper is opened up and broken down into cellulose fibers. Moreover, hemicellulose can be also obtained from the waste-paper or from the waste from the production of rice. This ensures e.g. a high tear resistance and tensile strength of the grip body. The fibers can also be primary fibers, e.g. of wood. A mixture of primary fibers and secondary fibers is also conceivable.

The share, a majority or all of the fibers, in particular of the natural fibers, in the grip body or in its material matrix are preferably aligned in its longitudinal direction. The alignment of the fibers in the longitudinal direction is preferably effected during the extrusion process.

The grip body or the material matrix, apart from the mentioned, primarily applied natural fibers, can yet include further fibers, in particular long fibers, with, for example, a greater average fiber length than the mentioned fibers, for the additional reinforcement of the grip body. The further fibers are preferably natural fibers, in particular vegetable fibers such as cotton fibers, hemp fibers, viscose fibers, jute fibers or mixtures thereof Cotton fibers or viscose fibers are preferably likewise secondary fibers which, for example, can be recycled fibers from the spinning industry or from waste-textile recycling.

The primarily applied fibers, in particular natural fibers can e.g. have lengths of greater or equal to 30 nm (micrometres), in particular of greater or equal to 0.2 mm. The mentioned fibers can moreover have lengths of smaller or equal to 8 mm, preferably of smaller or equal to 5 mm and in particular of smaller or equal to 2 mm.

The further fibers (long fibers) can have lengths of greater or equal to 2 mm, in particular of greater or equal to 5 mm The long fibers can have lengths of smaller or equal to 90 mm, in particular smaller or equal to 70 mm.

The grip body of the cotton bud preferably has a typical length of 5 to 10 cm. The diameter of the grip body is, for example, 0.5 to 3 mm, in particular 1 to 2 mm.

Both ends of the grip body designed as a small rod are enwrapped by cotton fluff if the grip body serves for manufacturing a cotton bud. The bud can be of plastic fibers or preferably also of biologically degradable natural fibers such as cotton.

The cross section of the rod-like body can be round (circular, oval, elliptical) or polygonal (triangular, quadrangular, rectangular or square, pentagonal, hexagonal, octagonal). Other cross-sectional shapes such as a combination of round and polygonal peripheral sections are also conceivable. The grip body can moreover be designed as a solid body or hollow body. A comparatively large scope for fashioning exists with regard to the cross-sectional shape of the grip body thanks to the application of an extrusion method.

Moreover, what is claimed is also a rod-like grip body, in particular for a cotton bud as described above. The grip body is preferably manufactured from a biologically degradable extrusion body. The grip body includes a material matrix as well as fibers, in particular natural fibers. The fibers preferably serve as reinforcement fibers. The grip body preferably includes fibers that are embedded into a material matrix.

All features of the grip body that are disclosed in this description, in the context of its use in cotton buds, also apply to the very generally claimed rod-like grip body. This is particularly the case also with regard to the nature of the material matrix and of the fibers, as well as the composite of the material matrix and fibers.

The rod-like grip body can be present as a hollow profile or solid profile.

The grip body can have a core, in particular a reinforcement core. The core is thereby preferably encased (coated or enveloped) by a material matrix. Embodiments concerning the nature of the material matrix have already been described further above. The material matrix can include fibers. Embodiments concerning the nature of the fires as well as concerning the composite/interconnection between the material matrix and fibers have likewise already been discussed further above.

The core is designed as a hollow body and is encased by a material matrix if the grip body is designed as a hollow body and this comprises the core.

The core can be a linear, textile formation. Embodiments concerning the nature of such a linear, textile formation as well as concerning the composite of the textile sheet formation to the material matrix have likewise already been discussed further above.

The core can moreover be a porous body with elastic characteristics. In other words, the core has a cell structure. The porous core in particular can be a foamed body or a rod. The body can be present as a hollow profile or a solid profile. The cell structure can be open-pored or closed-pored or mixed. The cells in particular enclose a gas.

It is also possible for the grip body to consist entirely of a porous body with, for example, elastic characteristics and in particular of a foamed body. In other words, the grip body has a cell structure. The cell structure can be open-pored or closed-pored or be mixed. The cells in particular enclose a gas.

If the grip body consists entirely of a body with a cellular structure as described above, then this can likewise be designed as a solid body or hollow body.

The foamed body can be foamed, for example, with the help of foaming agent such as water, CO₂ or N₂.

If the grip body has a foamed core or the grip body consist of a foamed material, then one can prevent a brittle breakage from occurring thanks to the nature of this material, by way of a deformation in the form of kinks and bulges taking place.

The subsequently described and claimed manufacturing method is to be generally applicable to rod-like grip bodies and not only grip bodies which are applied in cotton buds.

The grip bodies are preferably manufactured in an extrusion device. The extrusion device comprises a mixing device and an extrusion tool, via which the extrusion mass is extruded in a shaping manner. The method for manufacturing the grip body of a cotton bud includes the following steps:

extruding the extrusion mass through an extrusion nozzle amid the formation of an elongate extrusion body;

curing or solidifying the extrusion body.

The extrusion mass amongst other things comprises the later formed material matrix of the grip body.

According to a preferred embodiment of the invention, fibers, preferably biologically degradable fibers and in particular natural fibers, are mixed with a preferably biologically degradable matrix material in a mixing device, into an extrudable extrusion mass, before extruding the extrusion mass.

The extrusion mass can be highly viscous depending on the matrix. The extrusion mass can also be a dry mixture (dry blend).

The mixing or blending device can be integrated in the extruder. The fibers and matrix for this can be introduced into the extruder e.g. separately via two metering devices.

The components of the material matrix for example are fed in granulate form or in a fluid or flowable form to the extrusion device. The components must be melted as the case may be.

Water can be added during the mixing procedure, depending on which substances are applied and in which quantities. The addition of water can serve for the activation of the already present material matrix or of the binding agent such as lignin, gluten or starch. Moreover, the water addition can serve for the positive-fit cross-linking of fibers. The water addition can moreover serve for forming hydrogen bonds with a subsequent curing.

One can make do without the addition of water if the material matrix is a thermoplastic polymer or a mixture of plastic polymers, which for example relates to the listed biopolymers.

The extrusion mass is preferably mixed by way of an extruder screw. The extrusion mass can be premixed in a separate mixing device, the so-called compounder or mixer, even before the extrusion process. The grip body can accordingly be manufactured or extruded by way of a single-screw or double-screw extruder.

The extrusion body can be extruded horizontally or vertically, i.e. in the direction of gravity. The extrusion body exiting from the extrusion nozzle is cut to length into individual grip bodies before, during or after the curing.

Curing in the broadest sense means the solidification of the extrusion body. The feature of curing thus also includes the mere solidification of the extrusion body. In other words, the curing can include a mere drying, cooling and/or a polymerisation.

If the grip body includes a core, in particular a reinforcement core, as described above, then the grip body is preferably manufactured in a coextrusion method or encasing (coating method). Hereby, the extrusion mass which forms the later material matrix, and the core are led together and are commonly led through the extrusion nozzle or encasing nozzle.

The coextrusion method or encasing method is preferably likewise a continuous method with which the core, in particular the reinforcement core, is continuously fed as an endless longitudinal body to the extrusion tool or encasing tool and is unified or encased with the likewise continuously fed extrusion mass.

The rod like grip body, as has already been explained in detail, is preferably applied in cotton buds. The cotton buds according to the invention are applied in beauty care or cosmetics, for make-up, in baby care, when cleaning the ears. Moreover, cotton buds according to the invention are also applied as a so-called smear tool (for smearing saliva samples) such as for determining the genetic fingerprint with DNA mass screening.

The grip bodies according to the invention moreover can also be applied in the field of foodstuffs. The grip bodies can be for lolly sticks (lollypops, suckers, Chupa Chups). The grip bodies can also be sticks for the manufacture of ice cream on a stick.

Furthermore, matchsticks can also be manufactured from the grip bodies according to the invention.

The grip bodies can also be applied in further fields of body hygiene. Thus the grip body can also be applied in dental care, for example as a floss holder. A brush head or dental floss, for example, can also be applied at one end of the grip body instead of a bud. The grip body according to the invention can also serve as a holder for a tooth interstice cleaning element or for a tongue cleaner.

The grip body of the cotton bud according to the invention has the advantage that this has a comparatively high bending stiffness and simultaneously does not break in a splintering manner when the bending forces are high, but for example deforms plastically. Accordingly, the grip body according to the invention has plastic characteristics. Moreover, the grip body, as already mentioned, is inexpensive in its manufacture, in particular if recycled natural fibers are applied, as well as biologically degradable.

The features of the grip body that are disclosed in the context of the cotton bud can also be combined with the features of the independently claimed and disclosed grip body, and vice versa. Moreover, the features disclosed in the context of the manufacturing method can also be combined with the features of the cotton bud or grip body, and vice versa.

According to a further development of the invention, the grip body can also be treated with an active ingredient. The active ingredient can be applied onto the grip body in gaseous or liquid form. The grip body can thus, for example, be impregnated with the active ingredient.

The material can be a fragrant material, such as e.g. a perfume. In particular, grip bodies in the field of cosmetics can be treated with a fragrant material.

The active ingredient can also be have a medical effect, and i.e. act in a disinfecting manner.

### BRIEF DESCRIPTION OF THE DRAWINGS

The subject-matter of the invention is represented by way of embodiment examples according to the accompanying drawings.

There are shown in:

FIG. 1: a cotton bud;

FIG. 2: a particular embodiment of a grip body according to the invention;

FIG. 3: a further particular embodiment of a grip body according to the invention;

FIG. 4: a further particular embodiment of a grip body according to the invention;

FIG. 5: a further particular embodiment of a grip body according to the invention.

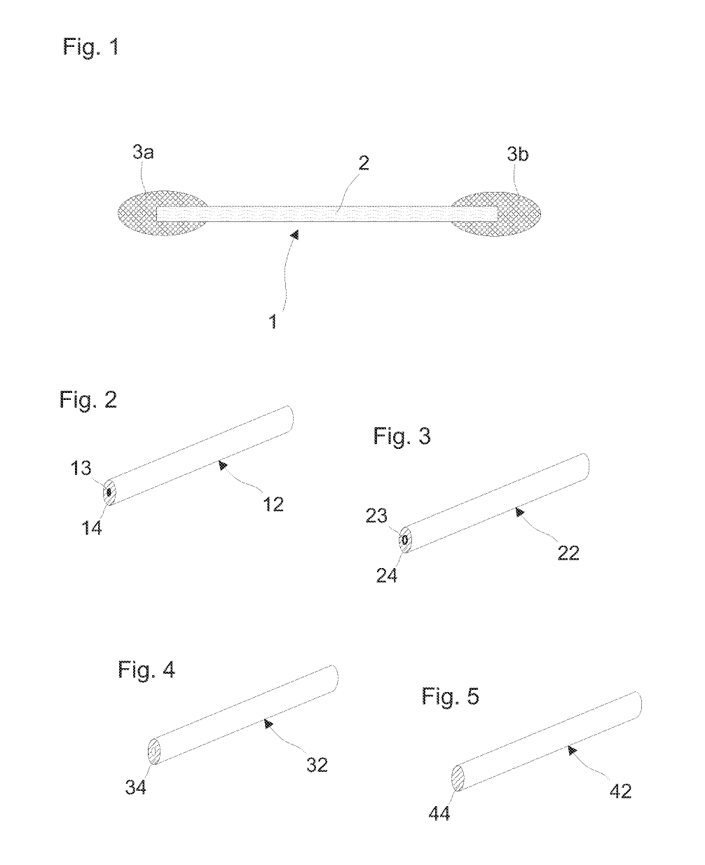

### DETAILED DESCRIPTION OF THE INVENTION

The shown cotton bud 1 consists of a rod-like grip body 2 in the form of a biologically degradable extrusion body, as well as of bud bodies 3a, 3b of cotton which are in each case wound around the grip body 2 at both ends.

The grip body 12 according to FIG. 2 has a reinforcement core 13 in the form of a thread of natural fibers that is enveloped by the material matrix 14 and is impregnated by this. The reinforcement core can also be a foamed body. The material matrix 14 can include fibers. The material matrix can be a foamed body with or without fibers.

The grip body 22 according to FIG. 3 has a fiber core 23 in the form of a tubular, textile body formation that is enveloped by the material matrix 24 and is impregnated by this. The fiber core can also be a tubular, foamed body. The material matrix can include fibers. The material matrix can also be a foamed body with or without fibers.

The grip body 32 according to FIG. 4 has a material matrix 34 and is designed as a hollow profile. The material matrix can include fibers. The material matrix can also be a foamed body with or without fibers.

The grip body 42 according to FIG. 5 has a material matrix 44 and is designed as a solid profile. The material matrix can include fibers. The material matrix can also be a foamed body with or without fibers.

1. A cotton bud with a rod-like grip body and with a bud body that is arranged at least at one free end of the grip.

2. The cotton bud according to claim 1, wherein the grip body comprises a reinforcement core in the form of a textile longitudinal body or a foamed core, which is surrounded by a material matrix.

3. The cotton bud according to claim 1, wherein the rod-like grip body is a biologically degradable extrusion body which comprises a biologically degradable material matrix as well as biologically degradable natural fibres, which are embedded into the material matrix, wherein the fibres are held together via the material matrix acting as a binding agent.

4. The cotton bud according to claim 1, wherein the material matrix comprises one of the following materials or a mixture of the following materials: starch, starch derivatives, sugar, lignin, chitin, gelatine, proteins, glycerine, fructose, sorbitol, cellulose derivatives, modified methyl cellulose, agar-agar, wax, pectin and/or biologically degradable polymers.

5. The cotton bud according to claim 3, wherein the grip body comprises 10 to 90% by volume, in particular 30 to 80% by volume, advantageously 40 to 70% by volume of fibres.

6. The cotton bud according to claim 3, wherein natural fibres are secondary fibres of a recycled material, such as waste paper, or from waste of rice production, or comprise such.

7. The cotton bud according to claim 3, wherein natural fibres are primary fibres or comprise such, and which are obtained directly from wood or wood waste.

8. A rod-like grip body for a cotton bud according to claim 1.

9. The rod-like grip according to claim 8, wherein the grip body is manufactured from a biologically degradable extrusion body.

10. The rod-like grip according to claim 8, wherein the grip body comprises a reinforcement core that is surrounded and/or is partly or completely impregnated, by the material matrix.

11. A method for manufacturing the grip body (2) according to claim 8 for a cotton bud according to claim 1, comprising the steps of:
extruding an extrusion mass forming the material matrix, through an extrusion nozzle or encasing nozzle amid the formation of an elongate extrusion body with a hollow profile or solid profile; and,
curing, such as solidifying or drying, the extrusion body.

12. The method according to claim 11, wherein the grip body comprises a reinforcement core, and the reinforcement core is fed as a longitudinal body and is co-extruded or encased together with the extrusion mass forming the material matrix.

13. The method according to claim 11, wherein natural fibres are mixed with a matrix material in a mixing device, into the extrudable extrusion mass, before extruding.

14. The method according to claim 13, wherein water is added to the extrusion mass during the mixing.

15. The method according to claim 11, wherein the extrusion body is cut to length into individual grip bodies before, during or after the curing.

16. (canceled)

17. A lolly with a grip body according to claim 1.

18. Ice-cream on a stick with a grip body according to claim 1.

19. A matchstick with a grip body according to claim 1.

20. A dental care product with a grip body according to claim 1.
